(19) United States
(12) Patent Application Publication
(10) Pub. No.: US 2007/0048359 A1
(43) Pub. Date: Mar. 1, 2007

(54) EXFOLIATING SCRUBBER

(76) Inventor: JOHN BOLTON, Santa Barbara, CA (US)

Correspondence Address: SoCAL IP LAW GROUP LLP, 310 N. WESTLAKE BLVD. STE 120, WESTLAKE VILLAGE, CA, 91362, US

(21) Appl. No.: 11/466,432

(22) Filed: Aug. 22, 2006

Related U.S. Application Data
(60) Provisional application No. 60/710,303, filed on Aug. 23, 2005.

(51) Int. Cl. A61K 9/70

(52) U.S. Cl. 424/443

(57) ABSTRACT

A scrubber mitt comprising a backing, a plurality of loops and a cavity. The backing may have an inner side and an outer side. The plurality of loops may be attached to the outer side. The cavity may be defined by the inner side. The cavity is adapted to fit around the hand of a person. There is also disclosed a process comprising disposing the scrubber mitt around a hand of a person and introducing a liquid cleanser into a reservoir defined by the backing.

### RELATED APPLICATION INFORMATION

This patent application claims priority from Provisional Application No. 60/710,303, filed Aug. 23, 2005, which is incorporated herein by reference.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### BACKGROUND

1. Field

This disclosure relates to cleansing devices.

2. Description of the Related Art

Wash cloths are commonly used in aiding the cleaning of a person's skin. Common wash cloths include synthetic and natural woven fibers. Additionally, people use luffahs and sponges to aid in the cleansing of a person's skin.

Wash cloths and wash towels tend to be inexpensive and are machine washable. Due to the abrasive and wet environment that such fabrics are exposed to, wash cloths and wash towels tend to fray, tear and break down. Moreover, because people tend to hand dry wash materials without sterilizing the material, bacteria, viruses, fungus, mold and other microorganisms tend to grow on the bathing material.

### DESCRIPTION OF THE DRAWINGS

FIG. 1 is an elevated view of a back of a scrubber mitt.

FIG. 2 is an elevated view of a front of a scrubber mitt.

FIG. 3 is a side plan view of a scrubber mitt.

FIG. 4 is an elevated partial cross sectional view 4-4 of the scrubber mitt of FIG. 3.

FIG. 5 is an elevated partial cross sectional view of 5-5 of the scrubber mitt of FIG. 4.

FIG. 6 is an elevated partial cross sectional view of 5-5 of the scrubber mitt of FIG. 4.

FIG. 7 is an elevated partial cross sectional view 7-7 of the scrubber mitt of FIG. 5.

FIG. 8 is an elevated partial cross sectional view 7-7 of the scrubber mitt of FIG. 5.

### DETAILED DESCRIPTION

Throughout this description, the embodiments and examples shown should be considered as exemplars, rather than limitations on the apparatus and methods disclosed or claimed.

Description of Apparatus

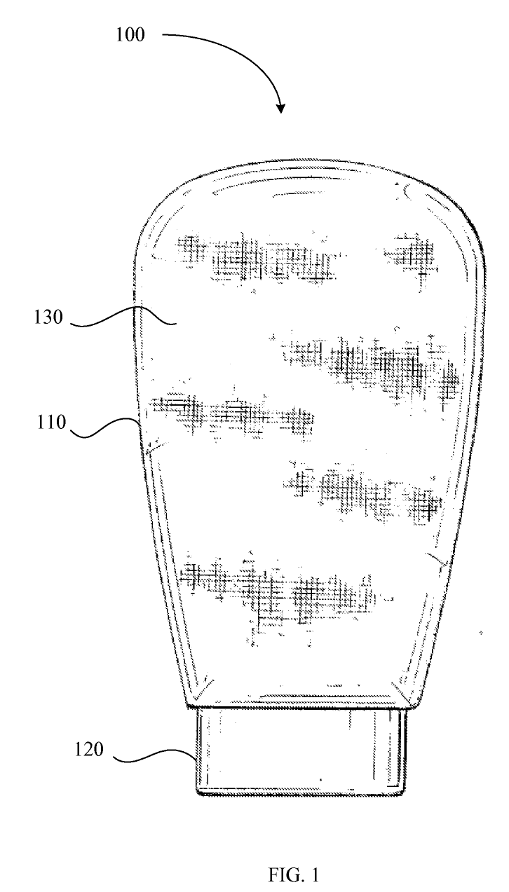

Referring now to FIG. 1 there is shown an elevated back view of a scrubber mitt 100. The scrubber mitt 100 may include a first section 110 and a second section 120.

The term scrubber mitt refers to a device adapted to fit over a person's hand having an abrasive first surface that is adapted to provide scrubbing and cleansing of surfaces of body parts in contact with the first surface and protection of the body part from attack by fungal or bacterial agents present on or on the first surface of the mitt. During scrubbing, dead skin cells may be removed from skin by abrading action of the first surface. Most of the dead skin cells may be removed from the first surface by flowing water on and/or through the first surface. Cells that remain on the first surface may be prevented from decay by fungal and bactericidal agents present in or on the first surface.

The first section 110 may have a generally rectangular geometry with beveled comers, generally oval shape, or other shape that is adapted to comfortably fit over the hand of a person. For example, the first section may have a length of approximately 9 inches, 11 inches, or other length, a width of approximately 5 inches, 9 inches, or other length, and a depth of approximately 3 inches, 4 inches, or other length. The dimensions of the first section 110 may be adapted for the intended user. For example, a large man may prefer a scrubber mitt 100 with a larger first section 110 than a small woman. Moreover, because a young child typically has a much smaller hand than a male adult, the scrubber mitt 100 may be manufactured in a variety of sizes.

A scrubber mitt 100 which is formed of resin based filaments may have a weight of approximately 1-10 pounds per square yard. Thus, children, adults and senior citizens may all easily pick up and hold the scrubber mitt 100. Moreover, dye may be added to the materials prior to or during the manufacturing process in order to provide colors, patterns and/or images that are appealing to children, adults and senior citizens. For example, a fluorescent color may provide for a feeling in a purchaser of being present in a relatively sterile UV environment.

The second section 120 may have a generally rectangular geometry with beveled corners or other shape. The second section 120 may be adapted to fit over the wrist of a person. In order for the scrubber mitt 100 to fit to a person's wrist, the second section 120 may include an elastic material which fits snugly with the person's wrist. Additionally, the second section 120 may include a snap, a strap, an adhesive and/or hook and loop fasteners in order for the second section 120 to fit snugly with the person's wrist.

The first section 100 may include a backing 130. The backing 130 may have the form of a panel. The term panel refers to a generally flat piece of material. For comfort and weight considerations, the backing 130 may have a thickness of approximately ⅕ inch to 1 inch or other dimensions. The backing 130 may be constructed from a water resistant or water proof material. Resistance to water helps reduce growth of bacteria, fungal, viral and other volatile organic compounds on the backing 130. Moreover, resistance to water helps the backing 130 maintain its shape and texture for a relatively long life cycle by resisting distortion due to water logging.

The backing 130 may comprise a material that is soft, pliable and/or yieldable. The backing 130 may have spring properties such that the backing 130 is comfortable to the hand. Examples of backing 130 materials include plastic, closed cell foam resin, sponge, rubber, combinations herein or other materials having similar characteristics.

The backing 130 may be porous or non-porous. Allowing water to escape from the surface proximate the backing aids in providing for an environment which bacteria and other microorganisms will not thrive.

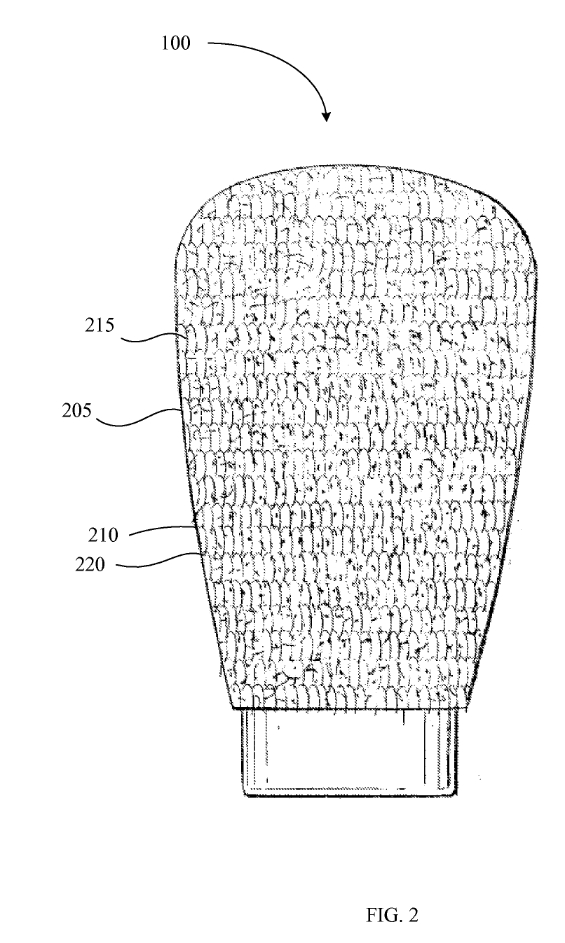

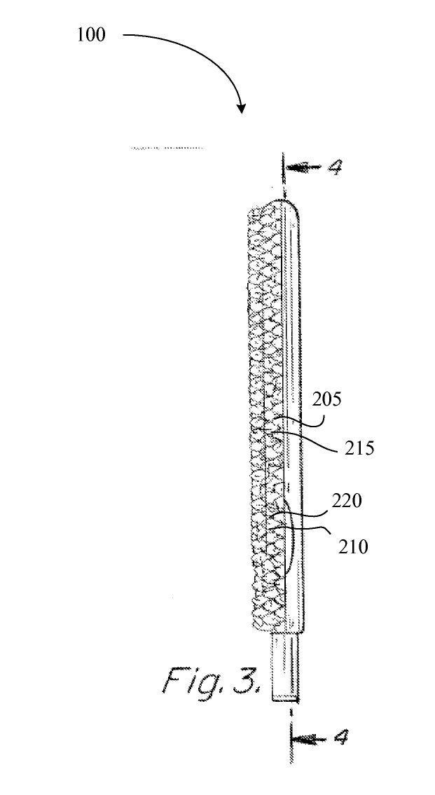

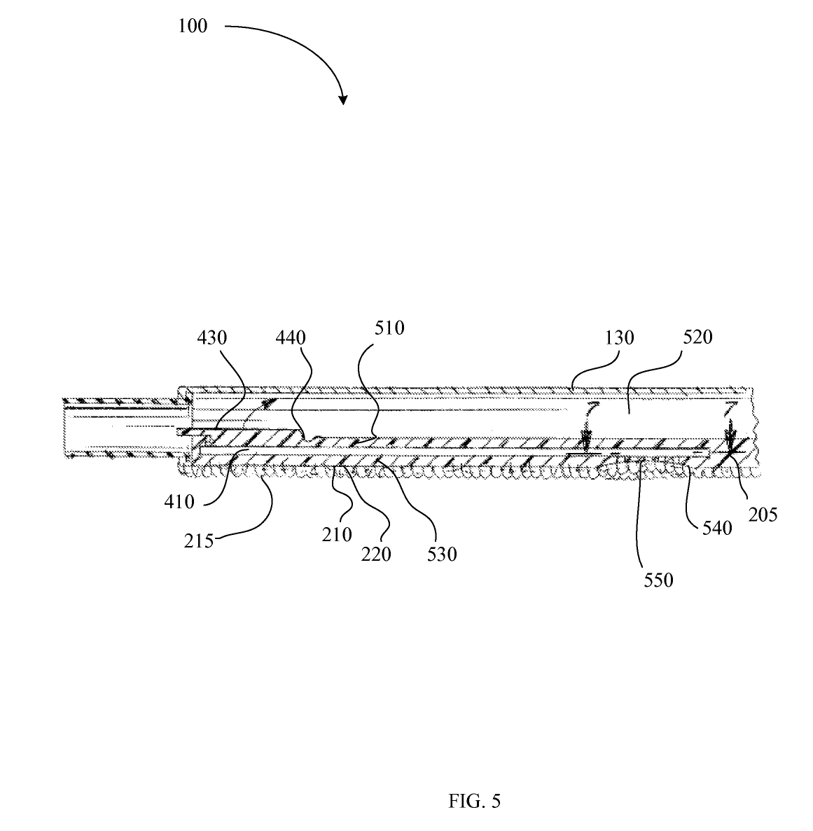

Referring now to FIG. 2, there is shown an elevated front view of the scrubber mitt 100 of FIG. 1. The front section 110 may include a backing 205 (not shown) and a plurality of loops 215. The loops 215 may have ends 210, 220 attached to or integrated with the backing 205 (as shown in the side plan view of FIG. 3 and the elevated partial cross sectional view of FIG. 5). The term loop refers to a ring or curved piece of material, such as a cone. The loop 215 may comprise a natural or synthetic fiber. The term fiber refers to a thin flexible single filament or a group of filaments twisted into a fiber having a relatively small cross section.

Example of loops 215 include a serpentine shaped synthetic resin, woven horsehair and stitched polyvinyl chloride, also known as PVC. The loops 215 may be manufactured from non-woven continuous or discontinuous lengths of filaments of organic resins. Organic resins may be thermoplastic, which aids in the spinning, crimping, shaping and bonding of filaments and/or fibers by thermal processes. Examples of organic resins include hydrocarbon resins such as vinyl, polyvinyl chloride (PVC), polyethylene, polyamides (Nylon), polyesters (Dacron) or polypropylene.

The loops 215 may be soft, yet have relatively high frictional qualities. The frictional qualities may be sufficient to abrade skin and exfoliate dead skin cells but not sufficient to scratch or irritate the skin. The selection of material may be based on a desired softness after treatment of a body parts, such as the back and neck, and the ability to easily exfoliate dead skin cells when one traverses the loops 215 across one's body parts. An example of the chemical composition of the loops 215 includes polymer at approximately 80% to 97% suitably by weight, vesicant at approximately 0% to 5% suitably by weight, inert mineral filler at approximately 1% to 10% suitably by weight, anti-fungicide at approximately 0.1% to 5% generally by weight and an anti-bacterial agent at approximately 0.01% to 3% usually by weight. An example of a polymer material is TVC, a polyvinyl chloride polymer at approximately 93.95%, vesicant at approximately 1%, clunch at approximately 4%, anti-fungicide at approximately 1% and an anti-bacterial agent at approximately 0.05%. An example of a chemical that acts both as an anti-bacterial and an anti-fungal agent is an oxy-substituted aromatic arsine such as oxybisphenoxarsine (OBPA).

The term mineral filler may include irregular lumps of rock such as clunch. Typically, clunch may include limestone, iron, chalk, and/or clay. Other fillers such as sand may be used. Clunch typically serves as a base for mortar.

The oxy-substituted aromatic arsine, such as OBPA, is a bacteriostat, disinfectant and fungicide. OBPA prevents the growth of microorganisms when compounded in resins and also acts as a preservative. OBPA can be formulated as an emulsifiable concentrate, pelleted, tableted, soluble concentrate/liquid and ready-to-use liquid.

The ends 210, 220 of the loops 215 may be integrated or bonded with the scrubber mitt 100 such that substantially none of the ends 210, 220 are free. A scrubber mitt 100 having loops 215 without any ends 210, 220 sticking out has aesthetic qualities as well as preventing a person from being scratched, poked and tickled by a free end 210, 220.

The loops 215 may be attached to or integrated with at least one side of the backing 205. The attachment of the loops 215 may be achieved via stitching, weaving, molding, bonding, adhesives or other methods. In turn, the loops may be embedded into the backing 205. Ends of the loops 215 may be bonded to the backing 205 and integrated with a closed cell foam material. In an embodiment of continuous filaments or fibers, the adhered or embedded bent lower end of a loop may be a starting point for the adjacent loop.

Referring again to FIG. 2, the loops 215 may be arranged side by side and/or may overlap each other. The loops 215 may be in contiguous rows and/or patterned mosaics. The loops 215 may be arranged randomly. The selection of the arrangement of the loops 215 may be determined for aesthetic reasons. The selection of the arrangement of the loops 215 may be determined for maximum frictional qualities in order to maximize dead skin cell exfoliation.

The loops 215 may have spring characteristics, or resiliency, such that when a person displaces the loops 215 in a variety of directions, the flattened loops 215 cushion the body part of the user and readily return or rebound to their original position relative the backing 205. For example, the loops 215 may have compressibility characteristics from approximately ½ to ¼ at 30 pounds per square inch. Moreover, the period for rebound may be approximately less than 1 second.

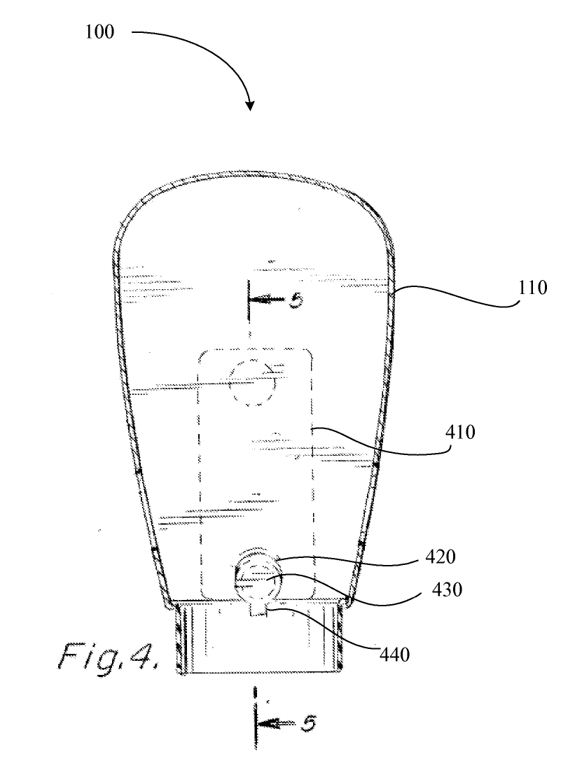

Referring now to FIG. 4, there is shown an elevated cross sectional view of 4-4 of the scrubber mitt of FIG. 3. As shown in cross hatches, the backing 205 of the first section 110 may include a reservoir 410, an opening 420, a cap 430 and a hinge 440.

The reservoir 410 is a part of the backing 205 in which a liquid may be at least partially contained. The reservoir 410 may be defined by the interior surfaces of the backing 205. The backing 205 may define an opening 420 into which liquid may be introduced into the reservoir 410. The reservoir 410 may have a regular or irregular shape. The dimensions of the reservoir 410 may span ½, ¾ or other proportion of the dimensions of the backing 205. The size and shape of the reservoir 410 may be selected based on the amount of liquid desired to be at least partially contained.

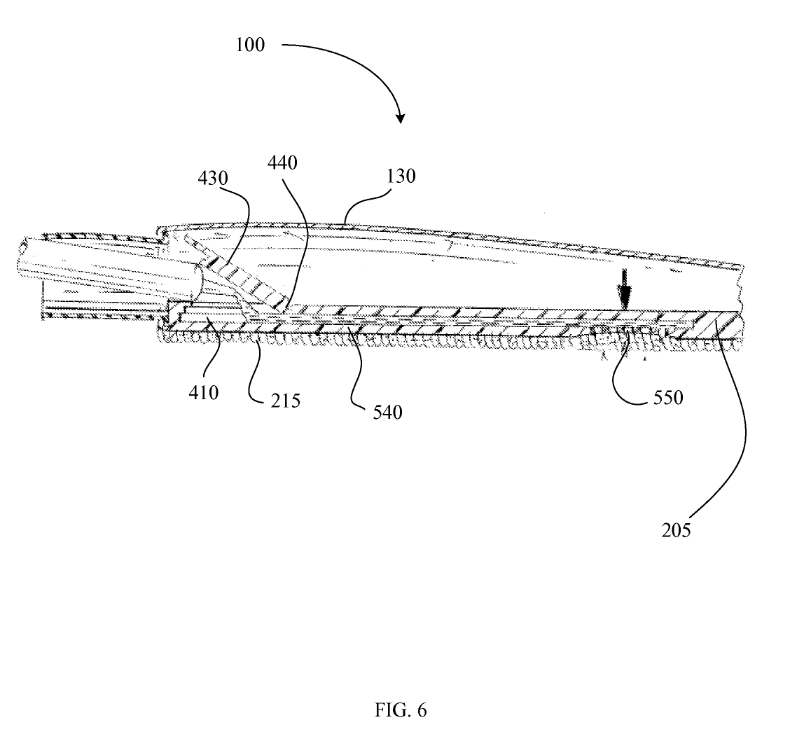

The reservoir 410 may include a cap 430. The term cap refers to a device that covers an opening such that a liquid does not pass through the opening beyond the cap. The cap 430 may be constructed of the same material as the backing 205 or a different liquid resistant material. The cap 430 may have dimensions based on those of the opening 420. The cap 430 may be integrated to the backing 205. The cap 605 may be attached to the backing 205 via a hinge 440 (as shown in the elevated partial cross sectional view 5-5 of the scrubber mitt 100 of FIG. 4 in FIG. 5 and the elevated partial cross sectional view 5-5 of the scrubber mitt 100 of FIG. 4 in FIG. 6).

Referring now to FIG. 5 and FIG. 6, the opening 420 may be disposed at any section of an inner portion 510 of the backing 205. The inner portion 510 is the portion of the backing 205 that is proximate a cavity 520 defined by the backing 205 and the backing 130. The size and dimensions of the cavity 520 may be selected based on the size of the user's hand or other dimensions.

The opening 420 may have a regular or irregular shape. The opening 420 may have a size that is selected based on dimensions just large enough to introduce liquids into the reservoir 410. For example, the opening 420 may have a generally circular shape with a 4 inch diameter or other shape and dimension.

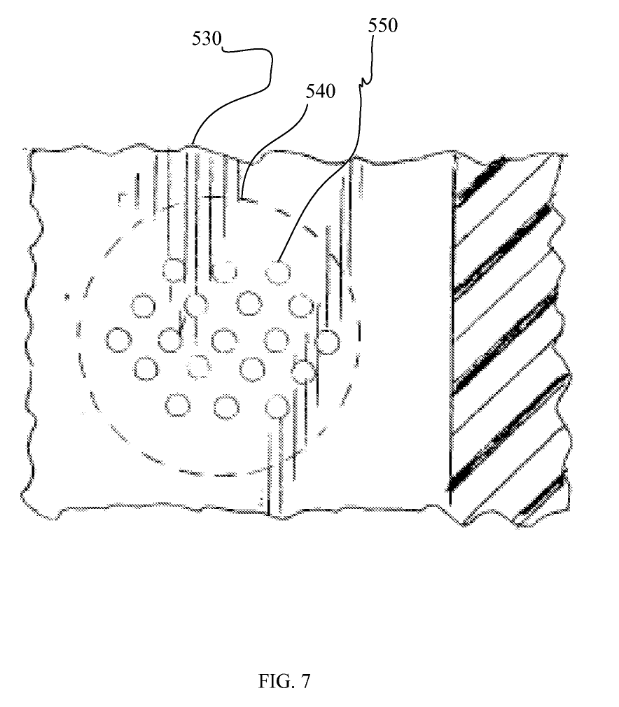

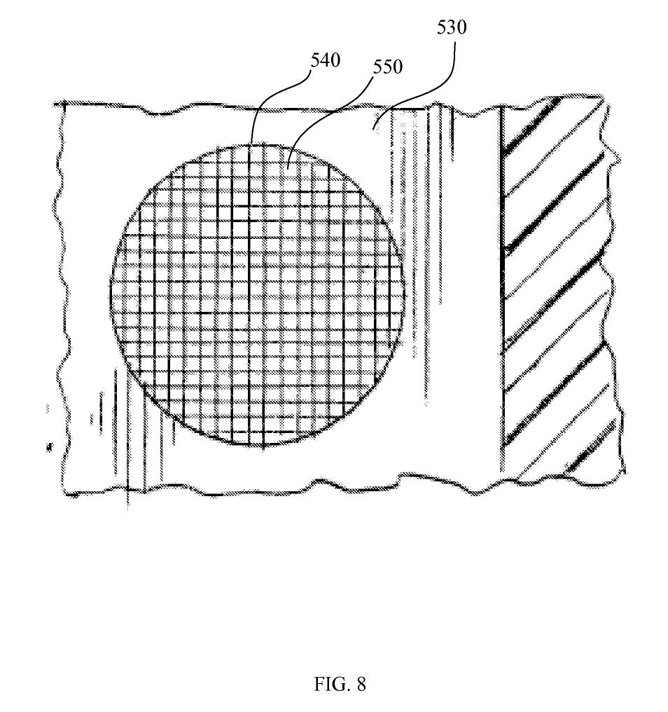

The backing 205 may include an outer portion 530 proximate the plurality of loops 215. The outer portion 530 and inner portion 510 may, in part, define the reservoir 410. The outer portion 530 of the backing 205 may include an opening 540. The opening 540 may include plurality of apertures 550 (also shown in the elevated partial cross sectional view 7-7 of the scrubber mitt 100 of FIG. 5 in FIG. 7 and FIG. 8). An aperture is a an opening or open space, such as a hole, gap, cleft, chasm or slit. The size, quantity and distribution of the apertures 550 may be determined based on the type of liquid for lathering and cleansing skin.

The apertures 550 may be adapted to remain impermeable to liquid within the reservoir 410 so long as a person does not rub the loops 215 across a person's body. The apertures 550 may be adapted to allow liquid to pass there through when a person applies force from the inner portion 510 of the backing 205 through the loops 215 to a body part.

If the liquid within the reservoir 410 is a cleanser, such as a liquid soap or another body lotion or medicinal cream, optionally including antifungal, germicidal and/or bacteriostatic agents, and a person rubs the loops 215 across their body, the liquid may be dispensed through the apertures 710, resulting in a lather on the person's body. The lather in combination with the abrasive qualities of the loops can be therapeutic to a person who works on their feet all day or generally enjoys self-pampering.

The scrubber mitt 100 may be used as follows:

(1) Remove cap 430 from the backing 205 to expose opening 420;

(2) Introduce liquid massaging lotion into the reservoir 410;

(3) Close opening 420 with cap 430;

(4) Apply force to the inner portion 510 of the backing 205 such that the reservoir 410 is squeezed as the loops 215 are pressed against a body part;

(5) Scrub the loops 215 against the body part;

(6) Clean the scrubber mitt 100 with water from the faucet.

The scrubber mitt 100 may be adapted to clean easier than luffah materials or sponges, resulting in reduced water consumption.

The relative position of various parts of the scrubber mat 100 are described herein based upon the elevated partial cross sectional view 5-5 of the scrubber mitt 100 of FIG. 4. For example, terms such as inner, outer, top, bottom, left and right are used. However, the scrubber mitt 100 may be used in various positions such as upside down for exfoliating the dead skin cells from a person's body part. Additionally, the scrubber mitt 100 may be used in a vertical position against a person's back for exfoliating the dead skin cells. Thus, some descriptive terms are used in relative terms and not absolute terms.

Closing Comments

The foregoing is merely illustrative and not limiting, having been presented by way of example only. Although examples have been shown and described, it will be apparent to those having ordinary skill in the art that changes, modifications, and/or alterations may be made.

Although many of the examples presented herein involve specific combinations of method acts or system elements, it should be understood that those acts and those elements may be combined in other ways to accomplish the same objectives.

As used herein, “plurality” means two or more.

As used herein, a “set” of items may include one or more of such items.

As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, “and/or” means that the listed items are alternatives, but the alternatives also include any combination of the listed items.

It is claimed:

1. An apparatus comprising:
a resin backing defining the shape of a mitt, the resin backing defining a reservoir, an opening disposed proximate an inner portion the resin backing and a plurality of apertures disposed proximate an outer portion of the backing
a plurality of loops bonded to the resin backing, wherein each of the plurality of loops comprises a resin material and a material selected from the group anti-fungal material and anti-bacterial material.

2. The apparatus of claim 1 wherein the resin backing includes a cap, the cap adapted to prevent liquid from passing through the opening.

3. The apparatus of claim 2 wherein the apertures are adapted to prevent liquid from passing through the apertures when a person is not rubbing the plurality of loops against a body part and allowing liquid to pass through the apertures when a person is rubbing the plurality of loops against a body part.

4. The apparatus of claim 3 wherein the plurality of loops comprises an anti-fungal material.

5. The apparatus of claim 4 wherein the plurality of loops each have a fluorescent color.

6. The apparatus of claim 5 wherein the anti-fungal material is OBPA and the resin backing includes a vinyl chloride resin including an aromatic arsine antifungal agent.

7. A process comprising:
disposing a scrubber mitt around a hand of a person, wherein the scrubber mat includes
a backing formed from an organic elastomer, the backing having a inner side and an outer side
a plurality of loops bonded to the outer side, each of the plurality of loops having an agent selected from the group consisting of an anti-bacterial agent and anti-fungal agent
the backing defining a reservoir, an opening disposed proximate the inner side of the backing and a plurality of apertures disposed proximate the outer side of the backing
introducing a liquid cleanser into the reservoir.

8. The process of claim 4 further comprising compressing the reservoir and rubbing the plurality of loops against the person thereby causing the liquid cleanser to pass through the plurality of apertures and lather.

9. The process of claim 9 further comprising closing the opening prior to disposing the scrubber mitt around a hand of a person.

10. The process of claim 9 wherein the opening is closed with a cap.

11. The process of claim 9 wherein the liquid cleanser is a body lotion.

12. The process of claim 11 further comprising exfoliating dead skin cells from the body of the person during rubbing the plurality of loops against the body.

13. A scrubber mitt comprising:
a backing having an inner side and an outer side,
a plurality of loops attached to the outer side,
a cavity defined by the inner side, wherein the cavity is adapted to fit around the hand of a person.

14. The scrubber mitt of claim 13 wherein the backing defines a reservoir, an opening disposed proximate the inner side and a plurality of apertures disposed proximate the outer side.

15. The scrubber mitt of claim 14 wherein the backing includes a cap, the cap adapted to prevent liquid from passing through the opening.

16. The scrubber mitt of claim 15 wherein the apertures are adapted to prevent liquid from passing through the apertures when a person is not rubbing the plurality of loops against the body of the person and allowing liquid to pass through the apertures when a person is rubbing the plurality of loops against the body.

17. The scrubber mitt of claim 16 wherein the plurality of loops comprises an anti-fungal material.

18. The scrubber mitt of claim 16 wherein the loops are resin filaments.

19. The scrubber mitt of claim 18 wherein the loops include anti-fungal agents.

20. The scrubber mitt of claim 18 wherein the attachment device comprises a magnetic material.

21. The scrubber mitt of claim 18 wherein the loops and the backing include a thermoplastic resin material.

22. The scrubber mitt of claim 21 wherein the thermoplastic resin material includes a bactericidal material.
